(19) United States
(12) Patent Application Publication
(10) Pub. No.: US 2010/0080826 A1
(43) Pub. Date: Apr. 1, 2010

(54) PROCESS FOR TREATMENT OF AMYOTROPHIC LATERAL SCLEROSIS, RHEUMATOID ARTHRITIS, TREMORS/PARKINSON'S DISEASE, MULTIPLE SCLEROSIS AND NON-VIRAL BASED CANCERS

(75) Inventor: George Nelson, Woodland Hills, CA (US)

Correspondence Address: DERGOSITS & NOAH LLP, Three Embarcadero Center, Suite 410, SAN FRANCISCO, CA, 94111, US

(73) Assignee: Salubrious Pharmaceuticals LLC, Los Angeles, CA (US)

(21) Appl. No.: 12/426,838

(22) Filed: Apr. 20, 2009

Related U.S. Application Data
(63) Continuation-in-part of application No. 12/298,904.
Application No. 12/298,904 is a continuation-in-part of application No. PCT/US08/11233, filed on Sep. 26, 2008.

(51) Int. Cl. A61K 39/13; A61P 37/04

(52) U.S. Cl. 424/204.1

(57) ABSTRACT

The present invention provides a composition and method for treating diseases associated with demyelination of the nerves, such as ALS, RA, Tremors/Parkinson's Disease, and MS, and for treating non-viral based cancers. By administering measured doses of an immunity-provoking agent and a bacterial antigen activator, patients suffering from ALS, RA, MS, Tremors/Parkinson's Disease, and prostrate cancer realized immediate beneficial results with no side effects.

### RELATED APPLICATION DATA

This nonprovisional patent application is a continuation in part of U.S. patent application Ser. No. 12/298,904, entitled “Process for Treatment of Rheumatoid Arthritis, Tremors/Parkinson's Disease, Multiple Sclerosis and Non-Viral Based Cancers,” filed on Oct. 28, 2008; which is the U.S. National Phase application of International Application Serial No. PCT/U.S.08/11775, filed Oct. 14, 2008; each of which are incorporated by reference in its entirety.

### FIELD

The present invention relates generally to the treatment of autoimmune disorders, and specifically, to the treatment of demyelinating diseases such as amyotrophic lateral sclerosis, rheumatoid arthritis, Tremors/Parkinson's Disease and multiple sclerosis. The present invention also relates to the treatment of non-viral based cancers.

### BACKGROUND

Rheumatoid arthritis (“RA”) is an autoimmune disease that is typically manifest by inflammation of the synovial joints. The development of RA progresses chronically, alternating between remission and relapse. Damage and deformation of joints can occur rapidly, particularly if the disease is untreated. As the disease progresses, RA can cause joint destruction, functional disability and premature mortality. RA can also include systemic inflammatory disease affecting multiple organs. RA patients often suffer physically and mentally from heavy pain all their lives. The cause of RA is presently unknown.

As an autoimmune disease, RA is characterized by a defect in the body's ability to distinguish foreign molecules from its own. The immune system attacks the synovial membrane, causing inflammation due to the infiltration of the membrane with T cells, plasma cells and macrophages. Formation of granulation tissue at the edges of the synovial lining is marked by extensive angiogenesis and enzyme production. These effects in turn cause progressive, erosive disintegration of adjacent cartilage and bone. In conjunction with the inflammation of the membranes, patients suffering from RA can also exhibit nerve abnormalities that primarily seem to involve segmental destruction of the myelin sheath.

Early stage prior art treatments typically attempt to ameliorate the pain symptoms through administration of non-steroidal anti-inflammatory drugs (NSAID). However, these treatments do little or nothing to affect the progression of RA.

Once a definitive RA diagnosis is made, conventional treatments include the use of steriods in conjunction with physical therapy and, if joint damage occurs, surgery. Again, these treatments have significant drawbacks and do not address the underlying causes of RA. For example, steroid therapy is associated with a number of well-known adverse side effects.

Specific compounds known as disease modifying anti-rheumatic drugs (DMARD) have been developed in an attempt to directly target the processes associated with RA. These DMARDs are typically administered in conjunction with NSAIDs. Examples of such compounds include Remicade®, methotrexate, and Humira®, which are all immunomodulators designed to inhibit the function of the body's immune system. While such treatments can slow the attack of RA, they undermine the ability of the immune system to respond normally to infections and leave the patient vulnerable to other diseases. Furthermore, they do not address the underlying causes of RA. Moreover, there are potentially severe side effects from using these immomodulators and there are restrictions placed on users to avoid exercise, alcohol and to be concerned about drug interferences.

As no cure for RA exists, there exists a need for treatments that alleviate the pain and inflammation associated with RA without the drawbacks inherent in prior art strategies. Similarly, there is a need for treatments that mitigate the joint damage associated with RA. One object of the current invention is to provide such treatments while minimizing the negative effects on a patient's immune system.

In addition to RA, there are a number of other progressive or degenerative diseases, such as Crohn's disease, multiple sclerosis (“MS”), Tremors/Parkinson's Disease, Alzheimer's disease, amyotrophic lateral sclerosis (“ALS”), Guillain-Barre syndrome, atherosclerosis, schizophrenia, Parkinsons's disease, senile dementia and others, associated with nerve damage. Although distinct, these diseases share common elements. Specifically, the precise origin or cause of these diseases remains unknown, yet they all exhibit damage to the nerves in the form of demyelination. As with RA, there is currently no cure for these diseases and prior art treatments have focussed on modulating the patient's immune system. For example, Copaxone® is administered to patients suffering from MS in order to suppress immune response. Naturally, a significant side effect of such treatments is the potential for the patient to have a compromised immune system.

Accordingly, there exists a need for treatments for MS, Alzheimer's disease, Parkinson's disease and the like that minimize the drawbacks associated with the prior art. Similarly, there is a need for a treatment for such diseases that helps prevent demyelination.

In certain cancers, there may be a latent viral infection that remains quiescent until some signal triggers a release from latency. Once triggered, the tumorous cell begins to replicate. The identification or disease etiology is difficult to assign because in some infections, the DNA of the causation virus is integrated into the genome of the host cell and is transmitted vertically. It therefore behaves as a genetic attribute. In other circumstances, the causative microbe triggers the cancer-disease process and then disappears from the body and is no longer detectable. What is needed, therefore is a vaccine that prevents single strand linear viruses from triggering the release of cancer from latency. It is these types of cancers, such as e.g., prostrate, liver, pancreatic, and lung cancer, that are referred to as the non-viral based cancers. Non-viral based cancers are to be contrasted with viral cancers whose etiology has been directly traced to viral causes. At present, only two viruses, human T-cell lymphotropic virus and human papillomavirus, are considered to be human tumor viruses. However, several other candidate viruses are implicated by epidemiological correlation, by serologic relationship or by recovery of virus from tumor cells.

The present invention satisfies these and other needs.

### SUMMARY OF THE INVENTION

The present invention is directed to composition useful in treating symptoms of diseases associated with demyelination of the nerves, such as ALS, RA, MS, Tremors/Parkinson's Disease and non-viral based cancers. In one embodiment of the invention, the composition includes an immunity-provoking agent and a bacterial antigen activator. Preferably, the immunity-provoking agent is a vaccine for a single-stranded RNA virus and more preferably, the immunity-provoking agent is an inactivated polio vaccine. Also preferably, the bacterial antigen activator is either or both tetanus toxoid and typhim VI.

Preferably, the composition comprises 5 parts of the inactivated polio vaccine to 1 part of the tetanus toxoid and 1 part of the typhim VI. Alternatively, the composition comprises 5 parts of the inactivated polio vaccine to 2 parts of either tetanus toxoid or typhim VI.

Also preferably, the composition is formulated for subcutaneous injection.

Another aspect of the invention is directed to a method for treating pain and inflammation in a patient comprising the steps of preparing a composition of an immunity-provoking agent and a bacterial antigen activator; and administering the composition to the patient. Preferably, the step of administering the composition comprises administering the composition subcutaneously. More preferably, the step of administering the composition comprises administering approximately 70 cc of the composition.

In one embodiment, the method includes treating a patient suffering from a demyelinating disease. Examples of such diseases include rheumatoid arthritis, multiple sclerosis, Alzheimer's disease, ALS, Guillain-Barre syndrome, atherosclerosis, schizophrenia, Tremors/Parkinsons's disease, and senile dementia.

In another embodiment, the method includes treating a patient suffering from a non-viral based cancer disease. Examples of such cancer diseases include prostrate cancers. The treatment of these disease conditions according to the compositions and methods of the invention eliminate the restrictions placed on the user's of prior art immunomodulators and the potentially severe side effects of these compounds.

### DETAILED DESCRIPTION

The present invention is a process for treating diseases associated with demyelination of the nerves, such as RA, MS, Alzheimer's disease, ALS, Guillain-Barre syndrome, atherosclerosis, schizophrenia, Tremors/Parkinsons's disease, and senile dementia, and for treating non-viral based cancers. By administering measured doses of an immunity-provoking agent and a bacterial antigen activator, patients suffering from these diseases and cancers have realized beneficial results. In connection with the non-viral based cancer diseases, the vaccination should be used, as appropriate, along with surgery, radiation and chemotherapy. However, as a vaccine, the present invention has the ability to combat the genesis of the non-viral cancer disease.

As discussed above, there exists a significant class of diseases for which the causative agents are poorly understood, but share a common symptom of nerve damage due to demyelination.

Myelin is the protective sheath around axons in the nervous system, also known as “white matter.” Myelin insulates the nerve and facilitates the conduction of the electrical potential associated with a neuronal signal. The myelin sheath is composed of glycolipids and proteins deposited around the axon by glial cells. Myelination of the nerves is an ongoing process that occurs during development and throughout childhood.

Demyelination can occur when the patient's immune system attacks the sheath, removing portions of the myelin from the axon. The physiological response to this damage causes the formation of gliotic plaques that interfere with conduction of the nerve impulses.

Without being limited to a particular theory, it is proposed that viral infection causes the patient's myelin to become targeted by the immune system. In response to the infection, the immune system produces antibodies to antigens associated with the infectious agent. However, when these antibodies are insufficiently specific and also recognize normal host antigens, such as components of the myelin sheath, a destructive, autoimmune response can result. Specifically, a dormant childhood infection could form the basis for a subsequent immune response that leads to one of the noted neurodegenerative diseases. Triggers for such a response could be severe physical/psychological trauma or it could be exposure to a suitable antigen or even the natural completion of the myelination process during the transition into adulthood.

In a related modality, a dormant childhood infection can also form the basis for triggering the replication of cancerous cells that have been in a latent state.

Accordingly, treatment with a suitable vaccine should counter this effect and compositions of the invention include an immunity-provoking agent.

Suitable immunity-provoking agents are preparations, such as vaccines, having the ability to confer a degree of immunity to a patient for a demyelinating disease. Preferably, the disease is also known to have the ability to penetrate the central nervous system (“CNS”) of the patient.

In one embodiment of the invention, the immunity-provoking agent comprises a polio vaccine. Poliomyelitis is a disease characterized by degradation of the myelin sheath, often leading to paralysis. The polio virus is a human enterovirus and member of the family of Picornaviridae composed of a single-stranded positive-sense RNA genome and protein capsid. Although a majority of polio infections are asymptomatic, in a small percentage of cases the virus does invade the patient's CNS, leading to the nerve damage that is the primary symptom of the disease. More preferably, the immunity-provoking agent comprises inactivated polio vaccine (“IPV”), such as trivalent IPV.

Other suitable uses for this vaccine with the single stranded RNA-based viruses that may be used in the practice of the invention include vaccines for rubella, mumps, measles, Rhinovirus virus, hepatitis A virus, Hepatitis C virus, Yellow Fever Virus, Dengue Virus and West Nile Virus.

It has been found that the compositions of the invention also require a bacterial antigen activator in conjunction with the immunity-provoking agent. Suitable bacterial antigen activators include gram-negative bacteria vaccines and gram-positive bacteria vaccines. Specific bacterial antigen activators found to be useful in the practice of the invention include tetanus toxoid and typhoid vaccine.

Clostridium tetani is a gram-positive, obligate anaerobic bacterium that produces the neurotoxin tetanospasmin. Tetanus toxoid is a modified form of tetanospasmin shown to stimulate the production of suitable antibodies and confer an immunity to tetanus. Salmonella enierica serovar typhi is a gram-negative, flagellated, rod-shaped bacterium and is the disease agent in typhoid fever. Typhoid vaccines are prepared from antigens particular to the bacterium. For example, the typhim VI vaccine is prepared from a cell surface polysaccharide of S. typhi.

The use of Diphtheria Toxoid to develop another vaccine to a single-strand virus is also intended to be within the scope of the invention.

Accordingly, in a presently preferred embodiment, the subject invention is directed to composition for subcutaneous injection comprising IPV, typhim VI and tetanus toxoid. More specifically, the composition of the invention preferably comprises 1 part tetanus toxoid, 1 part typhim VI, and 5 parts IPV. Alternatively, the composition comprises 2 parts tetanus toxoid and 5 parts IPV. In another alternative, the composition comprises 2 parts typhim VI and 5 parts IPV. The above ratios are all based on concentrations of IPV at (80 D antigen units Type 1)/mL, (16 D antigen units Type 2)/mL, and (64 D antigen units Type 3)/mL, tetanus toxoid at 10 Lf (flocculation units)/mL and 2 units antitoxin/mL, and typhim VI at 50 mg/mL.

The frequency and size of the vaccine dosage can be increased or decreased according to the patient's physical stature, and the general nature of the patient's health. However, preferably, the dosage remains at 70 cc per treatment.

For treatment in a patient suffering from pain and inflammation, the invention is a method comprising the steps of preparing a composition of immunity-provoking agent and bacterial antigen activator and administering the composition to the patient.

Preferably, the methods of the invention are directed to treatment of symptoms associated with RA, MS, Alzheimer's disease, ALS, Guillain-Barre syndrome, atherosclerosis, schizophrenia, Parkinsons's disease, senile dementia, and other diseases characterized by demyelization, and furthermore to the treatment of non-viral based cancers.

As noted above, the composition of the method preferably comprises 1 part tetanus toxoid, 1 part typhim VI, and 5 parts IPV, or 2 parts tetanus toxoid and 5 parts IPV, or 2 parts typhim VI and 5 parts IPV.

Also preferably, the step of administering the composition comprises subcutaneously injecting 70 cc of the composition.

A. Case Studies for Rheumatoid Arthritis (RA), Multiple Scleroses (MS), and Tremor's/Parkinson's (P), Prostrate Cancer (PC) and Amyotrophic Lateral Sclerosis (ALS).

Rheumatoid Arthritis (RA)

1. RA is a 61 year old male who has suffered from rheumatoid arthritis in his hands, fingers and back for the last 10 years. He started the medication 5 years ago and within one hour after taking the medication, the pain in his hands, fingers and back disappeared and by the second medication he continued to have no pain and no limitations of movement. He is basically symptom free of his rheumatoid arthritis and has continued taking the medication on a weekly basis. Absolutely no side effects.

2. RA is a 63 year old female who gave up golf as a result of rheumatoid arthritis. She has it in her hands, as well as her wrists and believes in her back for 10 years. She started the medication 2 years ago and within 45 minutes after the medication was administered, she was basically pain free, and had full and complete movement of both her wrists, hands and noticed no back pain whatsoever. She takes the medication once every 5 days and continues to remain pain free. Absolutely no side effects.

3. RA is a 82 year old man who had severe rheumatoid arthritis for 20 years. For the last 20 years both of his hands were clenched in a fist position and he suffered with severe pain in his hands. He received his first medication 3 years ago. After 45 minutes taking the medication he was crying for joy because this was the first time in 20 years he was without pain and an hour and a half after medication he was able to open his hands one inch. As his treatment continued every 5 days he regained full use of his hands with no pain and absolutely no side effects.

Multiple Scleroses (MS)

MS is a 62 year old female patient who has advanced MS. For eight years she suffered with severe pain in the right leg and was confined to a wheelchair, had incontinence, dysentery and multiple brain sheers (her doctor states that the last time she had seen a patient with this many brain sheers, it was a corpse). She started her medication 2½ years ago. Her first medication reduced her pain by 50% and the 2^{nd} medication 2 days later, within 45 minutes had no pain at all. The 3^{rd} medication 4 days later she was still pain free and was able to stand and use a walker to help her get around. The 4^{th} medication just 4 days later, she still showed no signs of pain, incontinence or dysentery and had no side effects. She began taking the medication every 5 days to maintain a healthy pain free life still with no dysentery and absolutely no side effects.

Tremor's/Parkinson's Disease (P)

P is a 64 year old man who noticed an occasional slight tremor in his left hand one year ago. He thought it was nerves. As time went on, the tremors were more frequent. He consulted with his doctor and was told it was it could be nerves or the beginning of Parkinson's Disease but there was no way to tell without an autopsy (not an option.) He tried compound vitamins, no help. After his first shot of the medication, the tremors stopped within 45 minutes, with no side effects. One week later, the left hand started some movement, I gave him another shot and the movement/tremors stopped. He has taken weekly shots since, and there have been no tremors and no side effects.

Prostrate Cancer

Twelve years ago P had a PSA score of 68 and a Gleason score of 7. A radical prostrate ectomy was performed, and P was given a prognosis of one to two years additional life. After P began administering the vaccination of the present invention, P's PSA score was −0.03 and has remained that way for twelve years.

Amyotrophic Lateral Sclerosis

YB is a patient that is in the final stages of ALS. She been on various pain medications over the years, but has not realized any significant pain abatement. Prior to receiving the vaccine, YB could not talk, her fingers were locked in a claw-like position, and she suffered from edema in her feet, legs, back and hand. Because of her pain, she was unable to move her jaw, thereby restricting her ability to eat. YB was also restricted from raising her arms above her chest due to the severe pain. In addition, YB suffered from shortness of breath, requiring an oxygen tank for breathing at night.

Vaccine was administered to YB four times per day. Within five days of continuous treatment, YB experienced significant reduction in her pain and edema. In addition, YB regained the ability to raise her arms and open her jaw. YB no longer needs an oxygen tank at night, and has regained the ability to speak.

One will appreciate that in the description above and throughout, numerous specific details are set forth in order to provide a thorough understanding of the present invention. It will be evident, however, to one of ordinary skill in the art, that the present invention may be practiced without these specific details. In other instances, well-known structures and devices are shown in block diagram form to facilitate explanation. The description of the preferred embodiments is not intended to limit the scope of the claims appended hereto.

What is claimed is:

1. A composition comprising an immunity-provoking agent and a bacterial antigen activator.

2. The composition of claim 1, wherein the immunity-provoking agent comprises a vaccine for a single-stranded RNA-based virus.

3. The composition of claim 2, wherein the immunity-provoking agent comprises an inactivated polio vaccine.

4. The composition of claim 1, wherein at least one bacterial antigen activator is selected from the group comprising tetanus toxoid and typhim VI.

5. The composition of claim 3, wherein the bacterial antigen activator is selected from the group comprising tetanus toxoid and typhim VI.

6. The composition of claim 5, further comprising 5 parts of the inactivated polio vaccine to 2 parts of the bacterial antigen activator.

7. The composition of claim 5, further comprising 5 parts of the inactivated polio vaccine to 1 part of the tetanus toxoid and 1 part of the typhim VI.

8. The composition of claim 1, wherein the composition is formulated for subcutaneous injection.

9. A method for treating pain and inflammation in a patient comprising the steps of:
preparing a composition of an immunity-provoking agent and a bacterial antigen activator; and
administering the composition to the patient.

10. The method of claim 9, wherein the step of preparing the composition comprises using a vaccine for a single-stranded RNA-based virus for the immunity-provoking agent.

11. The method of claim 10, wherein the step of preparing the composition comprises using inactivated polio vaccine for the immunity-provoking agent.

12. The method of claim 11, wherein the step of preparing the composition comprises using at least one bacterial antigen activator selected from the group comprising tetanus toxoid and typhim VI.

13. The method of claim 12, wherein the step of preparing the composition comprises using 5 parts of the inactivated polio vaccine to 2 parts of the bacterial antigen activator.

14. The method of claim 12, wherein the step of preparing the composition comprises using 5 parts of the inactivated polio vaccine to 1 part of the tetanus toxoid and 1 part of the typhim VI.

15. The method of claim 9, wherein the step of administering the composition comprises administering the composition subcutaneously.

16. The method of claim 15, wherein the step of administering the composition comprises administering approximately 70 mL of the composition.

17. The method of claim 9, wherein the patient is suffering from a disease characterized by demyelination.

18. The method of claim 17, wherein the disease is selected from the group consisting of rheumatoid arthritis, multiple sclerosis, Alzheimer's disease, ALS, Guillain-Barre syndrome, atherosclerosis, schizophrenia, Parkinsons's disease, and senile dementia.

19. A method for treating ALS in a patient comprising the steps of:
preparing a composition of an immunity-provoking agent and a bacterial antigen activator; and
administering the composition to the patient.

20. The method of claim 19, wherein the step of preparing the composition comprises using a vaccine for a single-stranded RNA-based virus for the immunity-provoking agent.

21. The method of claim 20, wherein the step of preparing the composition comprises using inactivated polio vaccine for the immunity-provoking agent.

22. The method of claim 21, wherein the step of preparing the composition comprises using at least one bacterial antigen activator selected from the group comprising tetanus toxoid and typhim VI.

23. The method of claim 22, wherein the step of preparing the composition comprises using 5 parts of the inactivated polio vaccine to 2 parts of the bacterial antigen activator.

24. The method of claim 22, wherein the step of preparing the composition comprises using 5 parts of the inactivated polio vaccine to 1 part of the tetanus toxoid and 1 part of the typhim VI.

25. The method of claim 19, wherein the step of administering the composition comprises administering the composition subcutaneously.

26. The method of claim 25, wherein the step of administering the composition comprises administering approximately 70 mL of the composition.

27. The method of claim 19, wherein the step of administering the composition comprises administering the composition four times a day.
